(19) United States
(12) Patent Application Publication
(10) Pub. No.: US 2008/0243036 A1
(43) Pub. Date: Oct. 2, 2008

(54) Spinal treatment method and associated apparatus

(76) Inventors: Dan Voic, Cedar Grove, NJ (US); Michael A. McManus, Bronxville, NY (US)

Correspondence Address: COLEMAN SUDOL SAPONE, P.C., 714 COLORADO AVENUE, BRIDGE PORT, CT, 06605-1601, US

(21) Appl. No.: 11/728,797

(22) Filed: Mar. 27, 2007

(51) Int. Cl. A61N 7/02

(52) U.S. Cl. 601/3

(57) ABSTRACT

Spinal pain is treated non-invasively by operating a scanning apparatus to locate a spinal disc afflicted with cracks or fissures, and applying waveform energy to the afflicted spinal disc to heat the spinal disc sufficiently to modify collagen fibers of the spinal disc and destroy pain receptors in the spinal disc. The scanning apparatus may be an ultrasound scanner, while the treatment waveform energy may be ultrasonic waveform energy. The applying of the waveform energy includes generating ultrasonic pressure waves in the spinal disc.

### BACKGROUND OF THE INVENTION

The present invention relates to method for treating certain kinds of spinal disease. More specifically, the present invention relates to a method of intradiscal heat therapy. The present invention also relates to an associated apparatus utilizable in the method.

Recent research has determined that a spinal disc may become painful as the disc annulus cracks and fissures, owing to natural degeneration or injury. These fissures in the disc annulus may become infiltrated with abnormal, pain-sensing nerve fibers and may allow inflammatory chemicals to leak into the spinal canal. Previously no treatment existed for chronically painful, degenerative discs short of major lumbar fusion surgery with removal of the painful discs and implantation of spinal hardware or bone.

Intradiscal endoscopic techniques, such as laparoscopic anterior lumbar interbody fusion, have been adapted for interior approaches to the lumbar spine. Although the endoscopic approach is promising, some limitations exist. Scientists have found the laparoscopic approach to involve longer operative times and a much higher rate of sexual dysfunction in men, whereas the open approach provides better visualization and is technically less demanding.

Intradiscal endoscopic treatment (IDET) is a new minimally invasive treatment for patients with low back pain caused by tears in the outer wall of one or more intervertebral discs. The therapy entails the application of heat to modify the collagen fibers of the degenerative disc and destroy the pain receptors in the area. An afflicted disc is heated by inserting an electrothermal catheter through which an electrical current passes.

IDET is performed as an outpatient procedure while the patient is awake and under a local anesthesia. The surgeon inserts the catheter through a small incision on the patient's back and into an afflicted disc under the guidance of an X-ray camera. Once in the disc space, the catheter heats the disc to a temperature of 90° C. over the course of about 20 minutes. The patient is observed for a while and then is allowed to go home. Pain relief may be seen within a few days following the procedure, or relief can take up to eight weeks to be noticed. Early studies indicate that in some patients the pain relief may continue for up to six months or longer. However, some patients do not experience any pain relief. The long-term effects of this procedure on the disc are not yet known.

Recovery from IDET takes one to two weeks. An exercise program after the procedure is often recommended. Early results with IDET show that some patients who undergo the procedure report an increased activity level, a reduced use of pain medications, and improved sitting tolerance. Later published results have been less positive. Long-term outcomes must be examined and compared to other forms of pain relief. More data into the effectiveness of IDET are needed especially in the form of placebo-controlled, randomized clinical trials.

The IDET's therapeutic functions are based on using heat to modify the disc's collagen fibers and destroying pain receptors in the target area.

### OBJECTS OF THE INVENTION

It is an object of the present invention to provide a method and/or associated apparatus for treating spinal pain originating intradiscally.

It is another object of the present invention to provide such a method and/or associated apparatus for treating spinal discs noninvasively.

A more specific object of the present invention is to provide such a method and/or associated apparatus for generating heat in a spinal disc to modify the disc's collagen fibers and destroy pain receptors in the target area

These and other objects of the invention will be apparent from the drawings and descriptions herein. Although every object of the invention is attained in at least one embodiment of the invention, there is not necessarily any embodiment which attains all of the objects of the invention.

### SUMMARY OF THE INVENTION

A method for treating spinal pain comprises, in accordance with the present invention, operating a scanning apparatus to locate a spinal disc afflicted with cracks or fissures, and applying waveform energy to the afflicted spinal disc to heat the spinal disc sufficiently to modify collagen fibers of the spinal disc and destroy pain receptors in the spinal disc. The waveform energy is generated outside the patient and travels through the patient's tissues to a focal point or other locus.

While the waveform energy may take any effective form, such as microwave or radio-frequency radiation, the waveform energy is preferably ultrasonic waveform energy. In that case, the applying of the waveform energy includes generating ultrasonic pressure waves in the spinal disc.

Pursuant to another feature of the present invention, the ultrasonic pressure waves are focused in the spinal disc, for instance, by operating a high-intensity focused ultrasound (HIFU) device. The HIFU transducer or wave generator module may comprise multiple transducer elements disposed in a fixed configuration of parabolic transverse cross-section that permits an optimization of the transducer's length/width ratio.

Pursuant to a further feature of the present invention, the scanning apparatus is an ultrasound apparatus. Accordingly, the operating of the scanning apparatus includes generating ultrasonic pressure waves in the spinal disc. Where both the scanning apparatus and the heat-inducing waveform generator are ultrasound devices, the devices may be separate dedicated devices. Alternatively, at least some transducer elements may be used to carry out both the imaging function and the therapeutic function. For instance, an ultrasound apparatus may include a multiplicity of transducer elements that are operated in a non-focused phased-array mode to extract image information that is processed to produce images that are displayed on a video monitor. Once an operating physician detects an afflicted spinal disc from the displayed images, the physician may operate the ultrasound apparatus to energize the phased transducer array so as to focus ultrasonic waves within the afflicted disc.

The waveform generating apparatus may include circuitry or programming for ensuring that a proper amount of ultrasonic waveform energy is applied to an afflicted disc. The control circuitry or programming ensures that enough energy is applied to heat the spinal disc sufficiently to modify collagen fibers of the spinal disc and destroy pain receptors in the spinal disc. The control circuitry or programming also ensures that the applied energy is limited to avoid overheating and consequent damage to the spinal disc collagen.

The scanning apparatus typically includes one or more electromechanical transducers, while the high-intensity focused ultrasound (HIFU) device includes at least one electromechanical transducer. Mounting structure may be provided for fixing the transducers of the scanning apparatus relative to the transducer of the HIFU device. The method of the present invention then further comprises moving the at least one second electromechanical transducer in tandem with the at least one first electromechanical transducer over a skin surface of the patient. The operating of the scanning apparatus includes energizing at least one electromechanical transducer to generate diagnostic ultrasonic pressure waves in the spinal disc, while the applying of the waveform energy includes energizing at least one other electromechanical transducer to generate therapeutic ultrasonic pressure waves in the spinal disc.

The transducers of the HIFU device may be dedicated elements, separate from the transducers of the scanning apparatus. This is likely to be the case where the treatment apparatus includes a probe having transducer elements fixed in a form conducive for wave concentration at a focal point or other locus. The treatment probe head may have its transducers disposed along a parabolic cylinder.

Alternatively, the HIFU device and the scanning apparatus may share transducer elements. This is possible, for instance, if the transducers are operated as a phased array first for imaging purposes to locate an afflicted spinal disc and subsequently for treatment purposes to heat the collagen material of the target disc.

Concomitantly, the treatment probe may include a dedicated set of transducers operated as a phased array, while the scanning apparatus includes another set of transducers operated separately as a phased array. Using such hardware, one may merely position the treatment probe and the scanning transducer array in juxtaposition to a patient's spinal cord at an approximate location of an afflicted or degenerative disc. Once the probe and the a scanning array are in place, the scanning and treatment may be effectuated without moving the transducers.

Alternatively, the scanning transducers as well as the treatment transducers may be located on a movable probe head. The probe is moved over a skin surface of the patient during a scanning procedure to locate an afflicted or degenerative disc. Once the disc is located, the probe head may be held in a fixed position during the application of focused waveform energy.

Accordingly, apparatus for treating spinal pain comprises, in accordance with the present invention, a waveform scanner adapted for locating a spinal disc afflicted with cracks or fissures, the waveform scanner including at least one sensor element disposable proximate to a patient. The apparatus further comprises a source of waveform energy for application to the afflicted spinal disc. The source includes a control circuit controlling the amount of applied waveform energy to heat the spinal disc sufficiently to modify collagen fibers of the spinal disc and destroy pain receptors in the spinal disc.

Where the waveform energy is ultrasonic waveform energy, the source includes at least one electromechanical transducer. The source includes means for focusing the ultrasonic waveform energy in the spinal disc. This means for focusing may take the form of a software program for energizing a plurality of spaced transducer elements in a phased array process. Alternatively, the means for focusing may include additional hardware, such as a multiplicity of piezoelectric transducers disposed in a parabolic array to generate high-intensity focused ultrasound.

In a particular embodiment of the present invention, at least a portion of the source of treatment waveform energy is fixed relative to the sensor and movable at in tandem with the sensor element relative to a skin surface of the patient.

The present invention provides a noninvasive method and associated apparatus for treating spinal pain originating intradiscally. The method and associated apparatus generate heat in a spinal disc to modify the disc's collagen fibers and destroy pain receptors in the target area

### BRIEF DESCRIPTION OF THE DRAWINGS

FIG. 1 is a block diagram of a system for treating spinal cord discs, in a method according to the present invention.

FIG. 2 is a block diagram of selected components of a control unit shown in FIG. 1.

FIG. 3 is a schematic cross-sectional view of an ultrasound treatment probe utilizable in a method in accordance with the present invention.

### DETAILED DESCRIPTION

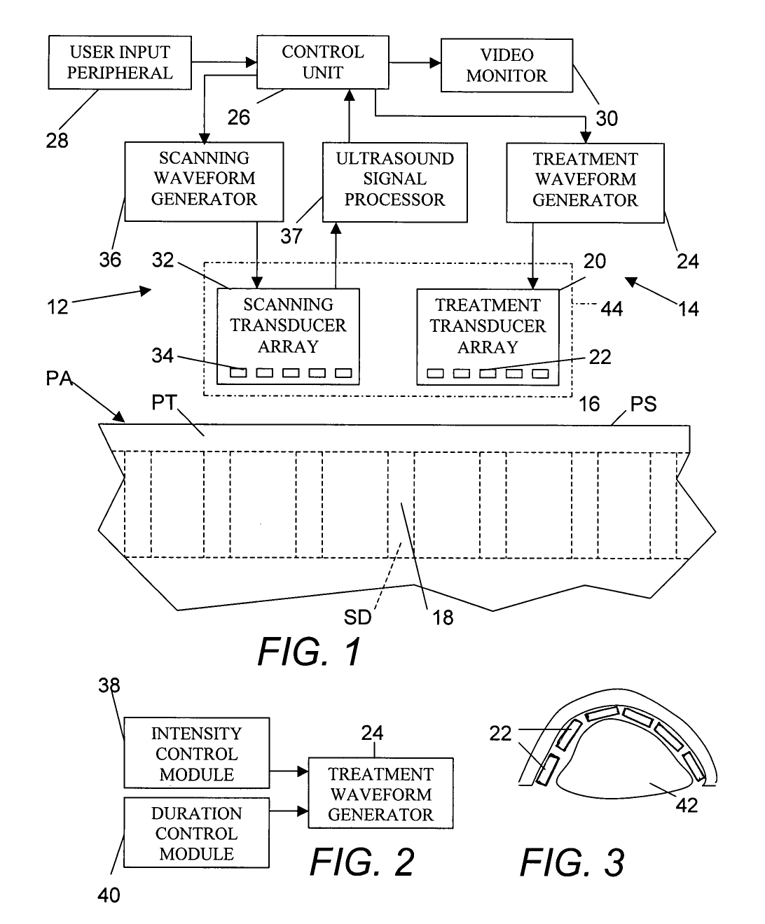

In a method for treating spinal pain, one operates a scanning apparatus 12 (FIG. 1) to locate, in a patient PA, a spinal disc SD afflicted with cracks or fissures. One then operates a treatment device 14 to apply waveform energy 16 to the afflicted spinal disc SD to heat the spinal disc sufficiently to modify collagen fibers of the spinal disc and destroy pain receptors in the spinal disc. The waveform energy 16 is generated outside the patient PA and travels through the patient's tissues PT to a focal point 18 or other locus.

Waveform energy 16 may take any effective form, such as microwave or radio-frequency radiation. Preferably, the waveform energy 16 is ultrasonic waveform energy. In that case, treatment device 14 comprises an array 20 of scanning transducers 22 that are placed into wave-transmitting contact with the patient's skin PS. Appropriate activation of transducers 22 generates ultrasonic pressure waves in the patient PA that are focused at point 18 in spinal disc SD.

Transducers 22 of array 20 are connected to a treatment waveform generator 24 that is in turn activated by a control unit 26 in response to instructions entered by a user via an input terminal or peripheral 28. During a scanning of a spinal column SC of the patient PA via scanning apparatus 12, the user views an image produced on a video monitor 30. Scanning apparatus 12 may take any convenient form (MRI, CAT) but preferably comprises an ultrasound scanner having an array 32 of transducer elements 34 that are selectively energized by a waveform generator 36 under the control of control unit 26. Transducer elements 34 may be piezoelectric crystals and are placed in wave-transmitting contact (e.g., using a gel) with the patient's skin surface PS generally over spinal column SC. Transducer elements 34 generate unfocused ultrasonic pressure waves in the patient's tissues PT that are partially reflected back to array 32. Transducers 34 are selectively polled by a signal processor 37 that conducts a preliminary processing of the incoming reflected waves and provides an analyzed or partially analyzed image-data-containing signal to control unit 26.

Control unit 26 modulates the operation of waveform generator 24 so that transducers 22 focus waveform energy 16 at point 16 in the afflicted or degenerative spinal disc SD to heat the spinal disc sufficiently to modify collagen fibers of the spinal disc and destroy pain receptors in the spinal disc. To that end, control unit 26 includes an intensity control module 38 and a duration control module 40 (FIG. 2) that regulate the amplitude and timing of the focused ultrasound. The therapeutic ultrasound radiation may be applied in pulses for better distribution and control. Intensity control module 38 and duration control module 40 cooperate to ensure that a proper amount of ultrasonic waveform energy is applied to an afflicted disc. The control circuitry or programming ensures that enough energy is applied to heat the spinal disc sufficiently to modify collagen fibers of the spinal disc and destroy pain receptors in the spinal disc. The control circuitry or programming also ensures that the applied energy is limited to avoid overheating and consequent damage to the spinal disc collagen.

Treatment device 14 may be a high-intensity focused ultrasound (HIFU) device. In that case, transducer elements 22 of treatment transducer array 20 are disposed in a fixed configuration of parabolic transverse cross-section (see FIG. 3) that permits an optimization of the transducer's length/width ratio. Reference numeral 42 represents a fluid-filled flexible pouch that facilitates the creation of an effective patient-probe interface over which ultrasonic pressure waves are conducted into the patient's tissues PT.

The operating of scanning apparatus 12 includes generating ultrasonic pressure waves in the target spinal disc SD. Where both scanning apparatus 12 and heat-inducing waveform-generating treatment device 14 are ultrasound devices, the devices may be separate dedicated devices. In that case the scanning arrays 20 and 32 may be mounted to respective substrates or carriers (not illustrated). Alternatively, at least some transducer elements 22, 34 may be used to carry out both the imaging function and the therapeutic function. In that case, scanning apparatus 12 and treatment device 14 are implemented via a single hardware arrangement. A common set of transducers, e.g., transducers 22 or array 20, perform the functions of transducers 22 and 34, while waveform generator 36 carries out the functions of treatment waveform generator 24, all in response to signals from control unit 26. In this combined functioning, transducers 22 may be operated in a non-focused phased-array mode to extract image information that is processed to produce images that are displayed on video monitor 30. Once an operating physician detects an afflicted spinal disc SD from the displayed images, the physician may instruct control unit 26 to energize the phased transducer array 20 so as to focus ultrasonic waves within the afflicted disc SD.

The common set of transducers may take the parabolic configuration illustrated in FIG. 3. Transducers 22 are energized according to different algorithms for imaging and therapy, respectively. In the case of therapy, the transducers are energized simultaneously to focus ultrasound simultaneously at the focal point 16 or other locus of the parabolic array 20. (For focusing at a point, the transducers are disposed along a parabola of revolution, while focusing along a line is implemented by a prismatic parabola configuration.) During scanning, the transducers 22 of FIG. 3 are energized one at a time and may also be polled in sequence.

Even where scanning apparatus 12 and heat-inducing waveform-generating treatment device 14 have respective dedicated transducer arrays 20 and 32, the arrays may be disposed on the same substrate, for instance, the same probe head 44, as diagrammatically illustrated in FIG. 1. Probe head 44 comprises mounting structure that fixes transducers 34 of scanning apparatus 12 relative to the transducers 22 of the HIFU device 14. Where the probe head 44 is movable by the operator over the patient's skin surface PS, the operator naturally moves treatment transducers 22 in tandem with the scanning transducers 34. Transducers 22 and 34 are typically electromechanical elements such as piezoelectric crystals.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A method for treating spinal pain, comprising: operating a scanning apparatus to locate a spinal disc afflicted with cracks or fissures; and applying waveform energy to the afflicted spinal disc to heat the spinal disc sufficiently to modify collagen fibers of the spinal disc and destroy pain receptors in the spinal disc.

2. The method defined in claim 1 wherein said waveform energy is ultrasonic waveform energy, the applying of said waveform energy including generating ultrasonic pressure waves in said spinal disc.

3. The method defined in claim 2 wherein the generating of said ultrasonic pressure waves in said spinal disc includes focusing said ultrasonic pressure waves in said spinal disc.

4. The method defined in claim 3 wherein the generating of said ultrasonic pressure waves in said spinal disc includes operating a high-intensity focused ultrasound device.

5. The method defined in claim 4 wherein said scanning apparatus is an ultrasound apparatus, the operating of said scanning apparatus including generating ultrasonic pressure waves in said spinal disc.

6. The method defined in claim 5 wherein said spinal disc is in a patient, said scanning apparatus including at least one first electromechanical transducer, said high-intensity focused ultrasound device including at least one second electromechanical transducer, further comprising providing mounting structure for fixing said at least one second electromechanical transducer relative to said at least one first electromechanical transducer, additionally comprising moving said at least one second electromechanical transducer in tandem with said at least one first electromechanical transducer over a skin surface of the patient.

7. The method defined in claim 1 wherein said spinal disc is in a patient and said waveform energy is ultrasonic waveform energy, the operating of said scanning apparatus including energizing at least one first electromechanical transducer to generate diagnostic ultrasonic pressure waves in said spinal disc, the applying of said waveform energy including energizing at least one second electromechanical transducer to generate therapeutic ultrasonic pressure waves in said spinal disc.

8. The method defined in claim 7, further comprising providing mounting structure for fixing said at least one second electromechanical transducer relative to said at least one first electromechanical transducer, also comprising moving said at least one second electromechanical transducer in tandem with said at least one first electromechanical transducer over a skin surface of the patient.

9. An apparatus for treating spinal pain, comprising: a waveform scanner adapted for locating a spinal disc afflicted with cracks or fissures, said waveform scanner including at least one sensor element disposable proximate to a patient; and a source of waveform energy for application to the afflicted spinal disc, said source including a control circuit controlling the amount of applied waveform energy to heat the spinal disc sufficiently to modify collagen fibers of the spinal disc and destroy pain receptors in the spinal disc

10. The apparatus defined in claim 9 wherein said waveform energy is ultrasonic waveform energy, said source including at least one electromechanical transducer.

11. The apparatus defined in claim 10 said source includes means for focusing said ultrasonic waveform energy in said spinal disc.

12. The apparatus defined in claim 11 wherein at least a portion of said source is fixed relative to said sensor and movable at in tandem with said sensor element relative to a skin surface of the patient.

13. The apparatus defined in claim 11 wherein said source is a high-intensity focused ultrasound device.

14. The apparatus defined in claim 10 wherein said scanner is an ultrasound device.

15. The apparatus defined in claim 14 wherein said at least one electromechanical transducer is a first electromechanical transducer, said scanner including at least one second electromechanical transducer, further comprising mounting structure fixing said at least one second electromechanical transducer relative to said at least one first electromechanical transducer.

16. The device of claim 15 wherein said scanner includes a handheld probe head, said at least one first electromechanical transducer and said at least one second electromechanical transducer being mounted to said probe head.
